(19) United States
(12) Patent Application Publication
(10) Pub. No.: US 2021/0190756 A1
(43) Pub. Date: Jun. 24, 2021

(54) SYSTEMS AND METHODS FOR EVALUATING CHARACTERISTICS OF ROPE UNDER USE

(71) Applicant: Samson Rope Technologies, Ferndale, WA (US)

(72) Inventors: James Plaia, Blaine, WA (US); Kris Volpenhein, Bellingham, WA (US)

(21) Appl. No.: 17/133,144

(22) Filed: Dec. 23, 2020

Related U.S. Application Data
(60) Provisional application No. 62/953,366, filed on Dec. 24, 2019.

(51) Int. Cl. G01N 33/36; D07B 1/14; G01N 3/56

(52) U.S. Cl. CPC G01N 33/367; D07B 1/145; D07B 2301/45; G01N 2203/028; G01N 3/56

(57) ABSTRACT

A non-destructive evaluation method for fiber rope comprises the following steps. A rope construction type is identified. An expected life of the rope construction type is determined. At least two characteristics of the rope construction types are identified. A characteristic adjustment factor is stored for at least one of the at least two characteristics. At least one rope characteristic interaction between at least two of the identified rope characteristics is identified. An interaction adjustment factor is stored for the at least one identified rope characteristic interaction. An adjusted remaining life is calculated based the expected life, the at least one characteristic adjustment factor, and the at least one interaction adjustment factor.

### RELATED APPLICATIONS

This application (Attorney's Ref. No. P220010) claims benefit of U.S. Provisional Application Ser. No. 62/953,366 filed Dec. 24, 2019, the contents of which are incorporated herein by reference.

### FIELD OF THE INVENTION

The present invention relates to systems and methods for evaluating characteristics of rope and, more specifically to rope evaluation systems and methods that may be used to assess characteristics non-destructively and, in many situations, without removing the rope from service.

### BACKGROUND

Quantitative non-destructive evaluation (NDE) of rope refers to the evaluation of rope characteristics indicative of ability of rope to serve a predefined function. In the following discussion, the term “fiber” will refer to non-metal natural and synthetic fiber structures, while the term “wire” will refer to metal structures.

Primarily due to the difference in the nature of the structure of fiber rope and wire rope, the effectiveness of NDE of fiber rope has lagged behind the effectiveness of NDE of wire rope. In particular, wire rope consists of a relatively small number (possibly several hundred for large ropes) of large diameter individual wire strands. It is relatively straightforward to interrogate the condition of each wire with an appropriate NDE technique and, from there, project the condition of the whole as the sum of the conditions of the components.

A large diameter fiber rope, on the other hand, may consist of hundreds of millions of micron-sized filaments. Fiber rope NDE systems and methods capable of interrogating or analyzing the condition of structures at microscopic size or that many filaments in a small area are not commercially available. Instead, fiber rope NDE has approached the problem by evaluating the apparent condition of higher-level aggregate structures, be it a yarn or strand within the rope, or the whole rope itself.

The most basic of these evaluations is a visual examination of the external surface of the rope. Added to this is an examination of the internal surfaces of the rope at discrete locations, when the rope structure and field use conditions allow such an examination. Evaluating external surface and/or internal surfaces at discrete locations using an empirical scale is known. Using this scale, a fiber rope can rate and/or record the condition of the rope as a single number or as two numbers: one number for an internal rating and one number for an external rating.

Commercially available fiber rope NDE systems and methods only report the condition of the rope from the standpoint of a single damage mode. Such systems and methods do not address other failure modes, particularly those without a visual cue, nor do they address the actual risk of failure during use associated with a rope in that reported condition. Using conventional fiber rope NDE systems and methods, each potential damage mode must be assessed separately to address such the actual risk of failure during use associated with a rope in that reported condition.

### SUMMARY

The present invention may be embodied as a non-destructive evaluation method for fiber rope comprises the following steps. A rope construction type is identified. An expected life of the rope construction type is determined. At least two characteristics of the rope construction types are identified. A characteristic adjustment factor is stored for at least one of the at least two characteristics. At least one rope characteristic interaction between at least two of the identified rope characteristics is identified. An interaction adjustment factor is stored for the at least one identified rope characteristic interaction. An adjusted remaining life is calculated based the expected life, the at least one characteristic adjustment factor, and the at least one interaction adjustment factor.

The present invention may also be embodied as a non-destructive evaluation system for fiber rope comprising a controller system, a data collection system for collecting data associated with fiber rope, a memory system, and a reporting system. The controller system stores in the memory system a rope construction type, an expected life of the rope construction type, at least two characteristics of the rope construction type, and at least one characteristic adjustment factor for at least one of the at least two characteristics, at least one rope characteristic interaction between at least two of the identified rope characteristics, and an interaction adjustment factor for the at least one identified rope characteristic interaction. The controller system further calculates an adjusted remaining life based the expected life, the at least one characteristic adjustment factor, and the at least one interaction adjustment factor.

The present invention may also be embodied as a non-destructive evaluation method for fiber rope comprising the following steps. A rope construction type is identified. An expected life is determined for the rope construction type. At least two characteristics of the rope construction type are identified. A characteristic adjustment factor us stored for at least one of the at least two characteristics. At least one rope characteristic interaction between at least two of the identified rope characteristics is identified. An interaction adjustment factor is stored for the at least one identified rope characteristic interaction. At least one characteristic adjustment amount is generated based on the at least one characteristic adjustment factor. At least one interaction adjustment amount is generated based on the at least one interaction adjustment factor. An adjusted remaining life is calculated based on the at least one characteristic adjustment amount and the at least one interaction adjustment amount.

### DESCRIPTION OF THE DRAWING

FIG. 1 schematically depicts an example rope under test;

FIG. 2 is a flow chart depicting initialization of a NDE system or method of the present invention;

FIG. 3 is a flow chart depicting use of a NDE system or method of the present invention;

FIG. 4 is a flow chart depicting use of a NDE system or method of the present invention to test multiple sections of an example rope under test; and

FIG. 5 is a block diagram representing an NDE system of the present invention.

### DETAILED DESCRIPTION

A rope evaluation system or method of the present invention evaluates each mode with at least one of a quantitative result that directly assesses the degree of risk associated with the current condition of that rope in that mode, a quantitative result that must then go through some correlation to assess the risk, or a qualitative result which must be correlated to some risk level. UV damage, for instance, is an example of a damage mode that typically lacks visual cues but assessment of which typically produces a quantitative result that can be directly related to changes in rope strength. Counting the number of cut strands in a rope is an example of a quantitative result which must then be related to rope risk through some correlation. Finally, an example of a qualitative measurement is the categorization of external or internal abrasion severity which can be empirically correlated to a risk level.

The overall risk analysis systems and methods of the present invention takes into account the fact that many of these modes have synergistic or antagonistic effects when forced to interact with each other due to proximity in the rope. The present invention thus includes a rope location-specific assessment over the full length of the rope such that potential interactions between damage modes might be evaluated. An example of such synergy in rope damage modes might include overall exposure of a rope to UV (which can weaken the polymer) coupled with abrasion over a length and a single cut strand within that length.

A further refinement permits inclusion of information derived from sources beyond the rope condition assessment. These information sources could include a time history of the tension applied to the rope during its use history, a count of bend cycles sections of the rope experienced, or even information that could be used to infer the rope history, such as the weather in the locations where the rope was used for mooring ropes because the weather conditions and mooring port locations affect the tensions and temperatures the rope experiences.

Finally, each damage mode evaluation to risk of rope failure correlation is a probabilistic assessment. Simple combinations will not give an accurate assessment; a combination of probabilities of failure and uncertainty in each evaluation must be made for an accurate picture for the rope health to result.

To address all of these complexities, a rope health risk analysis system or method of the present invention consists of separate and discrete evaluations that address, at the least, the major or primary damage modes that the rope might experience throughout its use. The evaluations may be applied along the length of the rope that received any type of damage in use. The reported conditions must then be imposed upon a virtual rope to determine if the possibility of synergistic or antagonistic behavior exists between the observed damage modes due to the nature and proximity of the modes. The resulting virtual rope combining all observations and potential combinatorial effects of those observations must then be analyzed with at least one correlation function, or possibly multiple correlation functions, to arrive at an estimate risk of failure for each region of rope along the length.

The resulting risk analysis can be used as a snapshot of the current condition and/or in conjunction with previous risk analyses prepared from earlier evaluations of the rope condition to understand the trend and the degree and nature of damage that a given rope use application might subject the rope to.

The resulting multi-sourced risk model can be used to provide a snapshot of the risk involved in using the rope or specific lengths of the rope at that point in time. If historical data is available, it can also be used to provide recommendations about retirement timelines in the future, even if the risk of rope failure is currently deemed acceptable.

For the purposes of this application, rope health may be defined as any single or combination of the following characteristics: retained strength after the rope has experienced field use; remaining time the rope may be put to use in the application, ignoring the non-working hours; remaining chronological time the rope is acceptable for continued use, including the non-working hours; retained axial stiffness of the rope (e.g., U.S. Pat. No. 10,288,538B2 patent shows that used ropes may show a loss of axial stiffness); and/or current assessment of other loss of continued functionality of the rope in the use application compared to a new rope.

The failure modes or damage types that might affect a rope include but are not limited to: abrasive damage to the external or internal surfaces of a rope; UV or other high energy damage to the base polymers used to make the synthetic rope fibers; fatigue damage, either at the rope structure level or at the base polymeric network level; thermal effects on the rope such as melting or degradation of strength due to increased temperature; and possible distortion of the rope structure such as from cut or snagged yarns or strands.

Each of these can be assessed individually for their ultimate effect on the health of an otherwise pristine and undamaged rope. Although difficult to probe or determine, synergistic or antagonistic effects of multiple damage modes interacting may be considered.

A specific example of a synergistic or antagonistic effect can be shown in the interrelationship between external abrasion and polymeric damage from UV exposure. In many commercially available ropes, the presence of pigment, added to color the rope fibers, can also serve to block penetration of UV energy into the rope structure. In a lightly abraded rope, the pigment added for this coloration can be abraded away and expose unpigmented fiber, thus the light abrasion and UV light exposure will serve together to decrease the remaining health of the rope more than would be anticipated from review of the two effects separately would suggest.

A very heavily abraded rope often shows a fuzzed appearance from the presence of loose fiber ends that extend out from the rope in all direction. This cloud of loose fibers around the rope partially obscures line of sight to the remaining load bearing fiber and thus absorbs a degree of any potential UV energy which would otherwise affect the polymer in the remaining load-bearing fiber. Thus, the heavily abraded state may form an antagonistic effect with UV exposure where the remaining rope health is higher than a review of the two individual effect separately would suggest.

This interaction is not restricted solely to damage existing within the rope. Environmental effects can lead to increased temperature through frictional or hysteretic heating of a rope that is undergoing motion or cyclic loading, respectively. External abrasion or a distorted rope structure might lead to greater frictional heating as the surface of the rope interacts to a greater degree with the substrate it is rubbing against compared to a pristine undamaged rope structure. The hysteretic heating of a rope undergoing cyclic loading is related to, among other effects, the fraction of the then-current rope strength to which the rope is being loaded. Thus, a rope that is otherwise weakened by other damage modes might show increased heating and increased loss of strength from that increased temperature.

Applied twist along the axis of the rope, beyond any twist applied during the manufacturing processes, can decrease strength and may interact with existing damage and/or environmental effects. Its effects, though, are reversible if the twist is removed. Furthermore, many uses of rope can lead to localized twist in particular regions of the rope even though other areas of the same rope do not show this increased twist.

A final example is the effect of creep or fatigue on a synthetic fiber rope. These two damage modes can cause damage that has no visible cue but still lead to compromised rope health. Instead, an analysis of the applied tension versus time history must be conducted for the rope in question. The lack of visual cue can lead to ropes which otherwise appear to have little visible damage exhibiting greatly diminished health due to interactions between the creep or fatigue damage and other damage modes that do have visual cues.

The creep and fatigue damage generally occurs at the level of the polymeric network and is a function of the stress applied on that network. Damage to the overall rope structure, of whatever type, weakens the rope and, if the tension applied to the rope remains constant, increases the stress applied to the remaining intact polymeric network. This leads to increased or accelerated creep or fatigue damage when compared to the rate that an undamaged rope would experience.

The effect on rope health for each of the damage modes mentioned has been evaluated, many only at the qualitative level but some at a quantitative or semi-quantitative level. The models or relationships are generally stand-alone and do not account for the presence of other damage modes. For a number of reasons, including the failure to account for other damage modes, difficulties in achieving an accurate damage assessment, and known variability in measuring the strength of rope; the resulting models or relationships all include a high degree of uncertainty. A simple accumulation of the various effects and levels of uncertainty would result in a compiled, multiple damage mode, model that had too high an uncertainty to be useful. If, for instance, a rope had at least three damage modes each decreasing the strength of the rope by 10% to 20%, a user of that model would find themselves unable to determine if the rope was still acceptable for use with 70% of the strength retained or requiring immediately replacement with 40% of the strength retained; 50% being usual accepted discard criteria for many industries.

In addition to the uncertainties discussed in damage assessment and damage effect, the synergistic/antagonistic effects of combinations of those damage modes are also a function of the proximity of those damage modes. Generally speaking, more proximate damage can produce a greater combinatorial effect. More distant damage tends to act as discrete effects with little interrelationship. This adds uncertainty from the necessity to report locations and extent of each damage type.

Statistically analysis can be used to address some of these uncertainties but the resulting treatment must address the physical realities of the rope structure. As ropes are twisted or braided constructions, they cannot, necessarily, be treated as a continuous structure. Further, the intermittent nature of ropes as strands that twist over each other or inter-braid with each other to produce the final structure should be considered when determining health of rope at any given point in time.

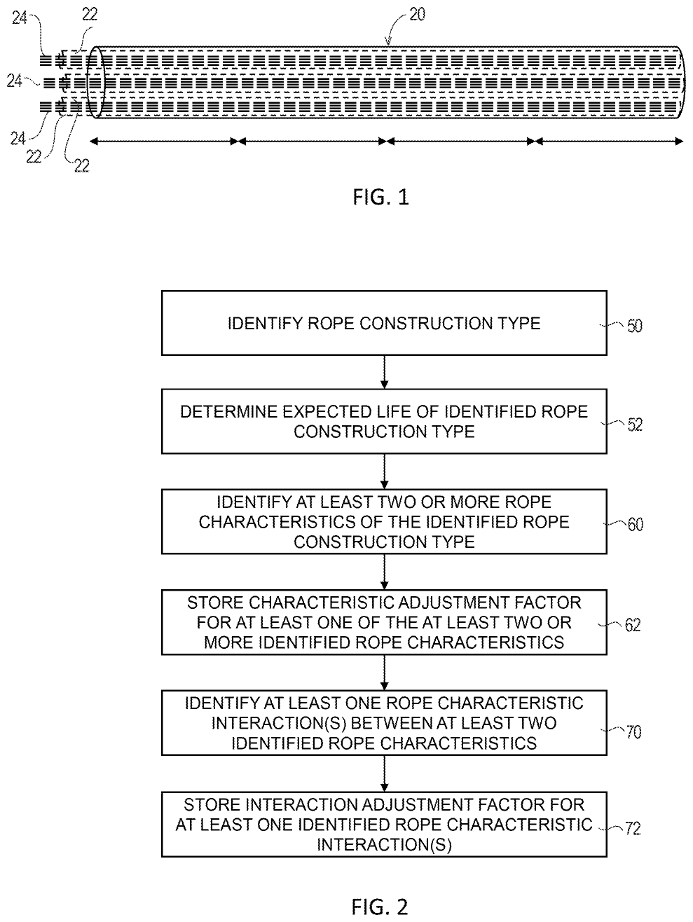

Referring now to FIG. 1 of the drawing, depicted therein is an example rope under test 20 that may be test using the NDE systems and methods of the present invention. As depicted in FIG. 1, the example rope under test comprises a plurality of rope sub-components 22 each comprising a plurality of rope fibers 24. While only one type of rope sub-component 22 is shown in FIG. 1, a particular type of rope can and typically will comprise more than one type of rope sub-component 22. For example, the rope fibers 24 are typically grouped in a first rope sub-component referred to as yarns, and the yarns are typically grouped in a second rope sub-component referred to as strands. In that case, the yarns are combined to form multiple strands and multiple strands are combined to form the rope. In addition, a particular rope may include fibers of different compositions, yarns of different sizes and compositions, and/or strands of different sizes and compositions. Further, the strands may be combined using any one or more techniques such as twisting and braiding, and the rope itself may comprises major components such as a core and a jacket. The example rope under test 20 thus is intended to generically represent ropes of different fiber compositions, yarn structures, strand structures, and rope structures.

FIG. 1 further illustrates that four different sections 30, 32, 34, and 36 of the example rope under test 20 may be identified. The health of each of these sections 30, 32, 34, and 36 may be determined separately and used to ascertain a total rope health score for the rope under test 20 as will be described in more detail below.

The NDE systems and methods of the present invention are configured and operated as follows. The systems and methods are first initialized using an initialization process as depicted in FIG. 2. In the initialization process, the NDE systems and methods of the present invention are first initialized for the rope composition and construction of a particular type of rope. For example, the type of rope composition and construction could be 12-strand braided rope made of a particular fiber.

Accordingly, at step 50 in FIG. 2 the rope construction type is identified, and an expected life for that particular rope construction type is determined at step 52. The expected life may be determined by means such as testing and/or based on modeling of the rope construction type.

At step 60, at least two or more rope characteristics of the identified rope construction type are identified. Examples of rope characteristics that may be determined at step 60 include any one or more of the example rope characteristics discussed above and any additional rope characteristics that may be determined through experience, testing, and additional investigation.

At step 62, a characteristic adjustment factor is identified for at least one of the two or more rope characteristics identified in step 60. The characteristic adjustment factor is or may be a numerical definition of the combined effect of rope use parameters (e.g., time duration, location, etc.) and rope characteristic on the expected life of the particular rope construction type. The numerical definition may be a single number or a set or table of numbers (e.g., a matrix) that can be used to represent the effect on rope life of the rope characteristic for a given set of operational conditions.

At step 70, at least one rope characteristic interaction of the identified rope construction type is identified. Examples of rope characteristic interactions that may be determined at step 70 include any one or more of the example rope characteristic interactions discussed above and any additional rope characteristic interactions that may be determined through experience, testing, and additional investigation.

At step 72, an interaction adjustment factor is identified for the rope character interaction(s) identified in step 70. The characteristic adjustment factor is or may be a numerical definition of the combined effect of rope use parameters (e.g., time duration, location, etc.) and rope characteristic interactions on the expected life of the particular rope construction type. The numerical definition may be a single number or a set or table of numbers (e.g., a matrix) that can be used to represent the effect on rope life of the rope characteristic interactions for a given set of operational conditions.

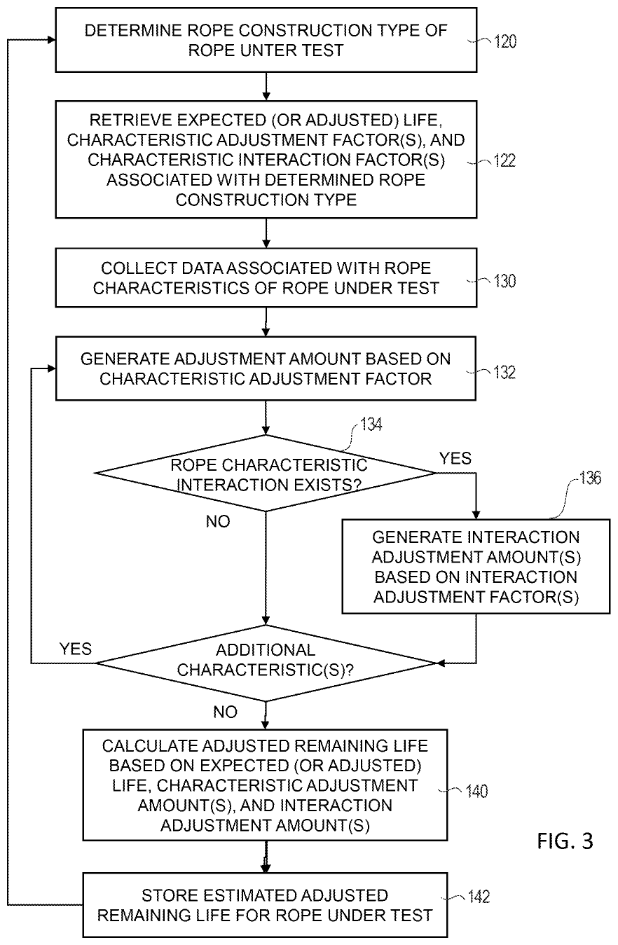

After the expected life, characteristic adjustment factor(s), and interaction adjustment factor(s) have been stored for a particular rope construction type, the system or method of the present invention is initialized and may be used to calculate an adjusted expected life for a particular piece of rope under test as shown in FIG. 3.

At an initial step 120, the rope construction type of the particular rope under test 20 is determined. Once the rope construction type is determined, expected (or adjusted) life, rope characteristic adjustment factor, characteristic interaction adjustment factor(s) are retrieved from storage at step 122 based on the rope construction type.

Next at step 130, data associated with the characteristics of the particular rope under test are collected. At step 132, an adjustment amount is generated based the characteristic adjustment factor of a first rope characteristic. At a step 134, the process determines whether at least one rope interaction exists for the first rope characteristic. If “YES”, at least one interaction adjustment amount is generated at step 136. If “NO” at step 134 or after step 136, the process proceeds to step 138, at which the process determines whether any additional rope characteristics exist. If “YES”, the process proceeds back to step 132 and repeats the process until no further rope characteristics exist. When no further rope characteristics exist (“NO” at step 138), the process proceeds to step 140.

At step 140, a new adjusted remaining life amount is calculated based on the expected or previously calculated adjusted life, one or more characteristic adjustment amount(s), and one or more interaction adjustment amount(s). As an example, if the remaining life at step 120 was 1200 hours, adjustment amounts of −20 hours, −120 hours, −200 hours, and +50 hours may be added to the original remaining life to obtain an adjusted remaining life of 910 hours. The adjusted remaining life is stored for that particular rope under test at step 142.

The process depicted in FIG. 3 is or may be repeated for a particular rope under test during the life of the rope. Accordingly, the process may return to step 120 after step 142 and be repeated as often as desired or required.

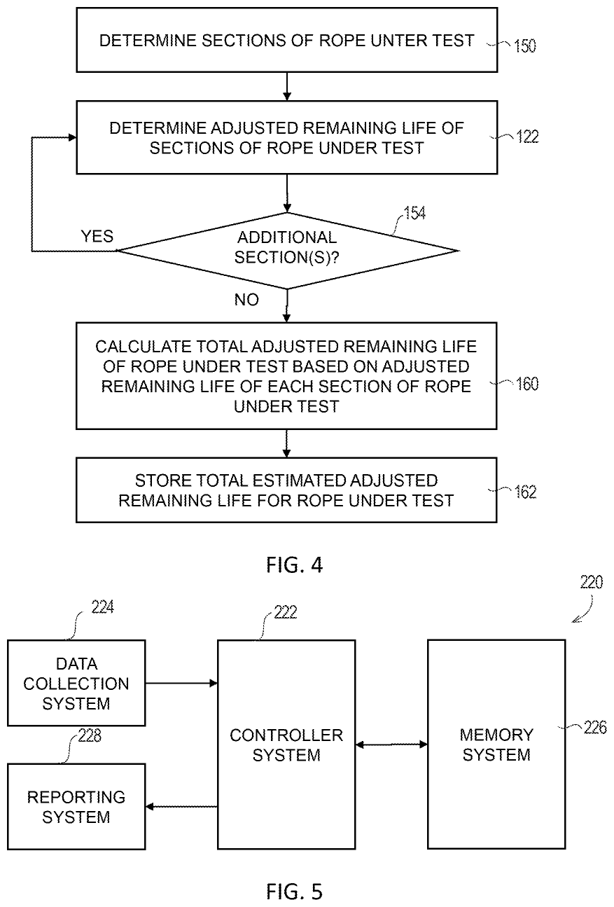

Referring now to FIG. 4, it can be seen that the process depicted in FIG. 3 can be performed for each of a plurality of the sections 30, 32, 34, and 36 as defined in FIG. 1 above. Although four sections 30, 32, 34, and 36 of substantially equal length are depicted in FIG. 1, more or fewer sections may be identified, and the sections may be of different lengths.

At a step 150, the process determines that the rope defines multiple sections each requiring a separate section health score. At step 152, an adjusted remaining life for a first of the sections of the rope under test is determined. At step 154, it is determined whether an adjusted remaining life needs to be calculated for any additional sections. If so, the process returns to step 152 and repeats until the answer at step 154 is “NO”.

When the answer is “NO” at step 154, the process proceeds to step 160 where a total adjusted remaining life of the rope under test is determined based on the adjusted remaining life of each of the plurality of sections identified in step 150. The total adjusted remaining life of the rope under test is stored at step 162.

If the total adjusted remaining life of the rope under test does not meet predetermined parameters, one or more of the sections 30, 32, 34, and 36 may be removed to improve the total adjusted remaining life, but for a shorter length of rope. The shortened length of rope may be used in the same environment or repurposed in an environment appropriate for the shorter length of rope.

FIG. 5 illustrates an example NDE system 220 of the present invention. A controller system 222 collects data from a data collection system 224, stores data in a memory system 226, and generates reports on a reporting system 228. The controller system 222, data collection system 224, memory system 226, and reporting system 228 may all be embodied as an app running on one or more general purpose portable computing device such as a smartphone, tablet computer, or laptop computer or may take the form a distributed computing system implemented by a number of separate devices, at least a portion of which is accessible over the internet (e.g., cloud-based computing).

In the example NDE system 220, the control system 222 implements the logic described above with reference to FIGS. 2-4, but this logic may be distributed throughout any one or more of the components 222, 224, and 228 of the NDE system 220 as appropriate for timely and secure implementation of this logic.

The data collection system 224 may be embodied as one, two, or more computing devices capable of allowing a user to obtain, record, or input data (e.g., numerical, visual, sound, spectral, chemical, etc.) indicative of rope characteristics processed by the example NDE system 220. Further, this data can be collected continuously, asynchronously, periodically, or according to a predetermined schedule by one or many computing devices over the life of the rope under test. The data collection system 224 may also take the form of a sensor system customized to collect and report, continuously, asynchronously, periodically, or according to a predetermined schedule, data such as weather conditions (e.g., thermometer, barometer), locations (e.g., GPS tracker), and use conditions (e.g., tension loads) of a specific rope under test. Such data can also be used to determine rope characteristics that may be considered by the example NDE system 220 when determining remaining life of a particular rope.

The example memory system 226 will typically take the form of a database capable of persistently storing data indicative of calculations of remaining life for a plurality of ropes under test. The database forming the memory system 226 will typically be configured to store data identifying and associated with many specific ropes in use at different locations. Typically, but not necessarily, the database forming the memory system 226 is configured to store, in addition to remaining life, all raw data associated with specific tests (e.g., locations, ambient conditions, rope characteristics, etc.) conducted at specific points in time for each specific rope tracked by the database forming memory system 226. The database forming the memory system 226 may be hosted by a third party such as Amazon or Microsoft and accessed by applications forming the components 222, 224, and 228 that are configured to run on the various computing devices forming the example NDE system 220.

The example reporting system 228 may also be embodied as one, two, or more computing devices capable of allowing a user to store, read, visualize, hear, or otherwise perceive reports indicative of the remaining life of a particular rope under test as calculated by the example NDE system 220. These reports can be read or distributed asynchronously, periodically, or according to a predetermined schedule by one or many computing devices over the life of the rope under test.

What is claimed is:

1. A non-destructive evaluation method for fiber rope comprising the steps of:
identifying a rope construction type;
determining an expected life of the rope construction type;
identifying at least two characteristics of the rope construction type;
storing a characteristic adjustment factor for at least one of the at least two characteristics;
identifying at least one rope characteristic interaction between at least two of the identified rope characteristics;
storing an interaction adjustment factor for the at least one identified rope characteristic interaction; and
calculating an adjusted remaining life based the expected life, the at least one characteristic adjustment factor, and the at least one interaction adjustment factor.

2. A method as recited in claim 1, further comprising the steps of:
generating at least one characteristic adjustment amount based on the at least one characteristic adjustment factor;
generating at least one interaction adjustment amount based on the at least one interaction adjustment factor; and
calculating the adjusted remaining life further based on the at least one characteristic adjustment amount and the at least one interaction adjustment amount.

3. A method as recited in claim 1, further comprising the step of collecting data associated with rope characteristics of a fiber rope under test.

4. A method as recited in claim 1, further comprising the step of determining the rope construction type corresponding to a fiber rope under test.

5. A method as recited in claim 1, further comprising the steps of:
identifying a plurality of sections of a rope under test; and
calculating the adjusted remaining life of each of the plurality of sections of the rope under test.

6. A method as recited in claim 5, further comprising the step of calculating a total expected remaining life based on the adjusted remaining life of each of the plurality of sections of the rope under test.

7. A non-destructive evaluation system for fiber rope comprising:
a controller system;
a data collection system for collecting data associated with fiber rope;
a memory system; and
a reporting system; wherein
the controller system
stores in the memory system a rope construction type, an expected life of the rope construction type, at least two characteristics of the rope construction type, and at least one characteristic adjustment factor for at least one of the at least two characteristics, at least one rope characteristic interaction between at least two of the identified rope characteristics, and an interaction adjustment factor for the at least one identified rope characteristic interaction; and
calculates an adjusted remaining life based the expected life, the at least one characteristic adjustment factor, and the at least one interaction adjustment factor.

8. A system as recited in claim 7, in which the controller:
generates at least one characteristic adjustment amount based on the at least one characteristic adjustment factor;
generates at least one interaction adjustment amount based on the at least one interaction adjustment factor; and
calculates the adjusted remaining life further based on the at least one characteristic adjustment amount and the at least one interaction adjustment amount.

9. A system as recited in claim 7, in which the controller further determines the rope construction type corresponding to a fiber rope under test.

10. A system as recited in claim 7, in which the controller further calculates the adjusted remaining life of each of a plurality of sections of the rope under test.

11. A system as recited in claim 10, in which the controller further calculates a total expected remaining life based on the adjusted remaining life of each of the plurality of sections of the rope under test.

12. A non-destructive evaluation method for fiber rope comprising the steps of:
identifying a rope construction type;
determining an expected life of the rope construction type;
identifying at least two characteristics of the rope construction type;
storing a characteristic adjustment factor for at least one of the at least two characteristics;
identifying at least one rope characteristic interaction between at least two of the identified rope characteristics;
storing an interaction adjustment factor for the at least one identified rope characteristic interaction;
generating at least one characteristic adjustment amount based on the at least one characteristic adjustment factor;
generating at least one interaction adjustment amount based on the at least one interaction adjustment factor; and
calculating the adjusted remaining life further based on the at least one characteristic adjustment amount and the at least one interaction adjustment amount.

13. A method as recited in claim 12, further comprising the step of collecting data associated with rope characteristics of a fiber rope under test.

14. A method as recited in claim 12, further comprising the step of determining the rope construction type corresponding to a fiber rope under test.

15. A method as recited in claim 12, further comprising the steps of:
identifying a plurality of sections of a rope under test; and
calculating the adjusted remaining life of each of the plurality of sections of the rope under test.

16. A method as recited in claim 15, further comprising the step of calculating a total expected remaining life based on the adjusted remaining life of each of the plurality of sections of the rope under test.
